(19) United States
(12) Patent Application Publication
(10) Pub. No.: US 2011/0077666 A1
(43) Pub. Date: Mar. 31, 2011

(54) LIGATING BAND DISPENSER DEVICE

(75) Inventors: Margaret McCAHON, West Lafayette, IN (US); Liana AUSTIN, Rancho Palos Verdes, CA (US); Louise Ginneh CORNELIUS, Medford, MA (US); Nancy DOREMUS, Cambridge, MA (US); Elizabeth KNEEN, Ottumwa, IA (US)

(73) Assignee: BOSTON SCIENTIFIC SCIMED, INC, Maple Grove, MN (US)

(21) Appl. No.: 12/876,629

(22) Filed: Sep. 7, 2010

Related U.S. Application Data
(60) Provisional application No. 61/247,285, filed on Sep. 30, 2009.

(51) Int. Cl. A61B 17/10

(52) U.S. Cl. 606/139

(57) ABSTRACT

A ligating band dispenser device and method for deploying ligating bands are disclosed. The device may comprise a ligating band support structure, at least one ligating band mounted around the outer surface of the ligating band support structure, a ligating band deployment structure, the ligating band deployment structure having an engagement structure for engaging and deploying the ligating bands, and a displacement mechanism adapted to displace one or the other of the ligating band deployment structure or ligating band support structure in an axial direction distally and proximally relative to the other structure.

### CROSS-REFERENCE

This application claims the benefit of U.S. Provisional Application No. 61/247,285 filed on Sep. 30, 2009, the disclosure of which is incorporated herein by reference in its entirety.

### FIELD OF THE INVENTION

The present invention relates generally to the field of tissue ligation, and more particularly to an improved device for dispensing ligating bands.

### BACKGROUND INFORMATION

Physicians have used elastic ligating bands to treat lesions, including internal hemorrhoids and mucositis and for performing mechanical hemostasis. For ligating tissue inside a body cavity, orifice, or lumen, physicians often use an endoscope to access the target tissue and ligate it. In one such form of endoscopic ligation, the physician uses the endoscope to position a stretched elastic band over the target tissue and then release the band onto the tissue so that the band contracts and catches the tissue.

The object of such ligation is to position a ligating band, which is usually elastic, over the targeted lesion or blood vessel section by first stretching the band beyond its undeformed diameter and then drawing the tissue to be ligated within the band. Thereafter the band is released so that it contracts, applying inward pressure on the section of tissue caught within the band. The effect of the inward pressure applied by the band is to cut off circulation through the targeted tissue, thereby causing the tissue to die. The body then sloughs off the dead tissue, or the dead tissue may be aspirated into an endoscope or a similar device.

Ligating instruments have been the subject of a number of patents, including U.S. Pat. No. 5,269,789 to Chin et al.; U.S. Pat. No. 5,356,416 to Chu et al.; U.S. Pat. No. 5,398,844 to Zaslaysky et al.; U.S. Pat. No. 5,857,585 to Tolkoff et al.; U.S. Pat. No. 5,853,416 to Tolkoff; U.S. Pat. No. 5,913,865 to Fortier et al.; U.S. Pat. No. 6,235,040 to Chu et al.; U.S. Pat. No. RE 36,629 to Zaslaysky et al.; and U.S. Pat. No. 7,063,709 to Fortier. The disclosures of these prior U.S. patents are expressly incorporated by reference herein.

U.S. Pat. No. 5,398,844 to Zaslaysky et al. allows a user to place several ligating bands at desired locations without removing the device from the patient's body to reload ligating bands. It uses multiple pull strings to deploy the ligating bands.

U.S. Pat. No. 5,913,865 to Fortier et al. (“the Fortier '865 patent”) describes a distal end for a ligating band dispenser that allows a plurality of ligating bands to be actuated sequentially by the same trigger line. The supporting structure as shown in the embodiments of FIGS. 1 and 17 of the Fortier '865 patent includes a plurality of slots in the distal end of the device that are arranged so that the trigger line need only pass through each slot once. The Fortier '865 patent describes how the slots can have alternating depths, such that alternating shallow slots and deeper slots are disposed on the distal end for retaining the trigger line.

### SUMMARY OF THE INVENTION

The invention is directed to improvements in ligating band dispenser devices and in methods for deploying ligating bands.

A disclosed embodiment provides for a ligating band dispenser device for mounting on a distal end of an endoscope comprising a ligating band support structure having a longitudinal axis, an outer surface, a proximal end and a distal end, with the ligating band support structure being adapted to be mounted on the distal end of the endoscope. The device also comprises one or more ligating bands mounted around the outer surface of the ligating band support structure, a ligating band deployment structure having an engagement structure for engaging and deploying the ligating bands, and a displacement mechanism adapted to displace the ligating band deployment structure in an axial direction distally and proximally relative to the ligating band support structure. In this embodiment, the ligating band deployment structure has a first engagement position in which the engagement structure engages a first ligating band, a deployment position distal to the first engagement position in which the engagement structure causes the engaged ligating band to deploy from the distal end of the ligating band support structure, and a second engagement position in which the engagement structure engages a second ligating band. The displacement mechanism displaces the ligating band deployment structure distally from the first engagement position to the deployment position to deploy the first ligating band. The displacement mechanism also displaces the ligating band deployment structure proximally from the deployment position to the second engagement position to engage the second ligating band, and the displacement mechanism displaces the ligating band deployment structure distally from the second engagement position to the deployment position to deploy the second ligating band.

Another embodiment provides for a ligating band dispenser device for mounting on a distal end of an endoscope comprising a ligating band support structure having a longitudinal axis, an outer surface, a proximal end and a distal end. The device also comprises one or more ligating bands mounted around the outer surface of the ligating band support structure, a ligating band deployment structure adapted to be mounted on the distal end of the endoscope and having an engagement structure for engaging and deploying the ligating bands, and a displacement mechanism adapted to displace the ligating band support structure in an axial direction distally and proximally relative to the ligating band deployment structure. In this embodiment, the ligating band support structure has a first engagement position in which the engagement structure engages a first ligating band, a deployment position proximal to the first engagement position in which the engagement structure causes the engaged ligating band to deploy from the distal end of the ligating band support structure, and a second engagement position in which the engagement structure engages a second ligating band. The displacement mechanism displaces the ligating band support structure proximally from the first engagement position to the deployment position to deploy the first ligating band. The displacement mechanism also displaces the ligating band support structure distally from the deployment position to the second engagement position to engage the second ligating band, and the displacement mechanism displaces the ligating band support structure proximally from the second engagement position to the deployment position to deploy the second ligating band.

Another embodiment provides a method of deploying a plurality ligating bands, comprising providing a ligating band dispensing device comprising a ligating band support structure having a longitudinal axis, an outer surface, a proximal end and a distal end, a plurality of ligating bands mounted around the outer surface of the ligating band support structure, a ligating band deployment structure having an engagement structure for sequentially engaging and deploying each of the ligating bands, and a displacement mechanism adapted to displace one of the ligating band deployment structure and ligating band support structure in an axial direction distally and proximally relative to the other of the ligating band deployment structure and ligating band support structure. In this embodiment, the method also comprises positioning the ligating band dispensing device in a first engagement position in which the engagement structure of the ligating band deployment structure engages a first ligating band mounted around the outer surface of the ligating band support structure, actuating the ligating band dispensing device from the first engagement position to a deployment position to deploy the first ligating band from the distal end of the ligating band support structure, positioning the ligating band dispensing device in a second engagement position in which the engagement structure of the ligating band deployment structure engages a second ligating band mounted around the outer surface of the ligating band support structure, and actuating the ligating band dispensing device from the second engagement position to the deployment position to deploy the second ligating band from the distal end of the ligating band support structure.

Depending on the embodiment, the invention can have advantages including simplicity of design, simplicity of manufacturing, reduced manufacturing costs, more reliable deployment, reduced complications during deployment, improved visibility, improved suction of tissue to be ligated, improved ease of use, avoidance of trigger line tangling or interference, improved room in the endoscope working channel, and/or improved overall performance. These and other features and advantages of the disclosed devices and methods are described in, or apparent from, the following detailed description of various exemplary embodiments.

### BRIEF DESCRIPTION OF THE DRAWINGS

Various embodiments will be more readily understood through the following detailed description, with reference to the accompanying drawings, in which:

FIG. 1 is a perspective view of a ligating band dispenser device according to a first embodiment;

FIG. 2 is a perspective view of a ligating band dispenser device according to a second embodiment;

FIG. 3 is a perspective view of a ligating band dispenser device according to a third embodiment;

FIG. 4 is a perspective view of the ligating band dispenser device of FIG. 1 with the outer cylinder in a resting position;

FIG. 5 is a perspective view of the ligating band dispenser device of FIG. 1 in the first engagement position;

FIG. 6 is a perspective view of the ligating band dispenser device of FIG. 1 moving from the first engagement position to the deployment position;

FIG. 7 is a perspective view of the ligating band dispenser device of FIG. 1 after the ligating band has been deployed;

FIG. 8 is a schematic of the flexible engagement element of the first and second embodiments shown in FIGS. 1 and 2;

FIG. 9 is a schematic of another embodiment of a flexible engagement element;

FIG. 10 is a schematic of another embodiment of a flexible engagement element;

FIG. 11 is a schematic of another embodiment of a flexible engagement element;

FIGS. 12a-12c are perspective views of three embodiments of a ligating band dispenser device mounted on an endoscope with a trigger wire mechanism for retracting the outer cylinder for dispensing the ligating bands;

FIG. 13 is a schematic of one embodiment of an exemplary actuator for dispensing the ligating bands;

FIG. 14 is a schematic of another embodiment of an exemplary actuator for dispensing the ligating bands; and

FIG. 15 illustrates an alternative embodiment in which the outer cylinder is fixed relative to the endoscope and the inner cylinder is movable relative to the endoscope to deploy the ligating bands.

### DETAILED DESCRIPTION

For a general understanding of the features of the illustrated embodiments of the invention, reference is made to the drawings. In the drawings, like reference numerals have been used throughout to designate like elements.

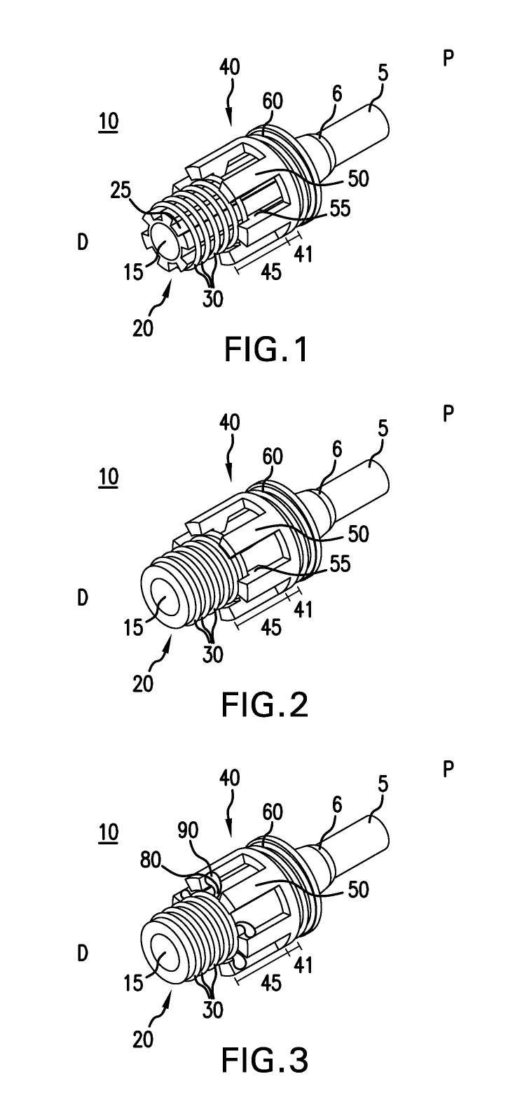

As illustrated in FIG. 1, a ligating band dispenser 10 according to a first embodiment includes a ligating band support structure in the form of a substantially cylindrical inner structure 20 which has a central bore or channel 15 extending axially through the inner structure 20. The central channel 15 is wide enough to accept tissue into the channel and allow visualization through it, for example, when the ligating band dispenser 10 is attached to the distal end of endoscope 5. As illustrated, the reference letter P indicates the proximal side of the device, and the reference letter D indicates the distal side of the device. In this embodiment, the ligating band dispenser 10 includes a flexible connector 6 which allows the ligating band dispenser 10 to be attached to the distal tip of endoscope 5. A plurality of elastic ligating bands 30 are received in a stretched condition around the inner structure 20.

Inner structure 20 has a longitudinal axis extending from a proximal end to a distal end. Inner structure 20 also has an outer surface. As illustrated in FIGS. 1-3, ligating bands 30 are mounted around the outer surface of inner structure 20 by stretching the ligating bands 30 onto and around the outer surface. Ligating bands 30 may be held on the outer surface by the compressive force of the elasticity of the bands or by any means known conventionally in the art including, but not limited to, transverse ridges or grooves on the outer surface of the inner structure as disclosed in, for example, U.S. Pat. No. 7,063,709 to Fortier.

As illustrated in FIGS. 1-3, a ligating band dispenser 10 according to these illustrated embodiments also includes a ligating band deployment structure in the form of an outer structure 40 positioned outside the outer surface of inner structure 20. In these embodiments, outer structure 40 comprises two main parts, a mounting portion 41 and an engagement structure 45. As illustrated in FIGS. 1-3, the shape of the outer structure 40 may be substantially cylindrical and configured to surround the outer surface of inner structure 20 in a circumferential direction.

The engagement structure 45 extends distally in an axial direction from the mounting portion 41 of the outer structure 40. The engagement structure 45 is used to sequentially engage and deploy each of the plurality of ligating bands 30, as described further below. In the embodiments of FIGS. 1-3, the engagement structure 45 comprises flexible engagement elements 50 in the shape and form of arms that extend distally from the mounting portion 41. The flexible engagement elements 50 further comprise an engagement surface 55 that, as the device is used, sequentially abuts and engages each of the plurality of ligating bands 30. In use, as described further below, the engagement surface 55 pushes each of the ligating bands 30 distally to deploy each of the ligating bands 30 from the distal end of the inner structure 20.

According to the first embodiment, the inner structure 20 also comprises at least one track 25 extending in the direction of the longitudinal axis of inner structure 20. As illustrated in FIG. 1, inner structure 20 may comprise a plurality of tracks 25, and each track 25 may accommodate a flexible engagement element 50. In this embodiment, each flexible engagement element 50 has a corresponding track 25 allowing movement of the flexible engagement element 50 within its respective track 25 to guide the flexible engagement element 50 along the axial direction of the inner structure 20 during engagement and deployment of the ligating bands 30. The depth and shape of the tracks 25 are not particularly limited, but may generally be configured to the shape of the portion of the flexible engagement element 50 that travels within each track. The tracks 25 facilitate the engagement and deployment of the ligating bands 30 by allowing the engagement surface 55 to engage the ligating band 30 below the inner radial surface of the ligating band 30, which enhances the rolling or sliding action of the ligating band 30 as its moves distally along the outer surface of the inner structure 20.

FIGS. 2 and 3 illustrate a second and third embodiment, respectively, wherein the ligating band support structure or inner structure 20 does not have tracks. In these embodiments, the innermost radial surface or edge of the flexible engagement elements 50 rests or contacts the inner structure 20 on the outer surface. In these embodiments, at least a bottom portion of the engagement surface 55 engages the ligating band directly and pushes the ligating band distally as it slides or rolls off the distal end of the inner structure 20.

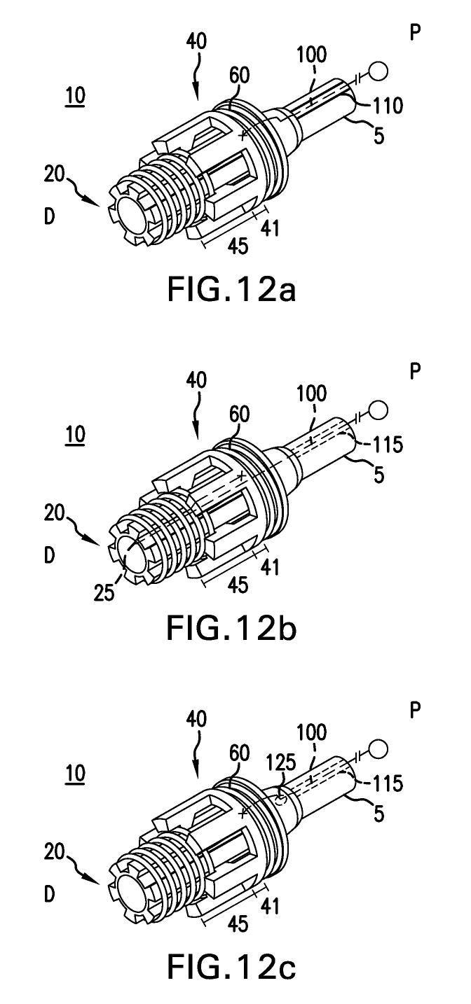

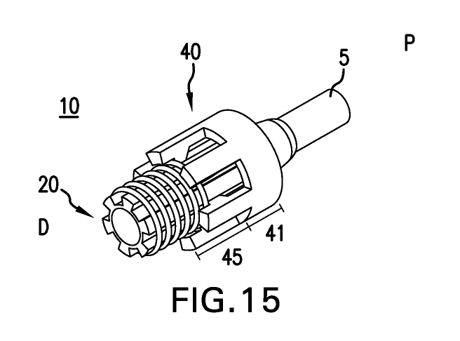

The ligating band dispenser 10 also comprises a displacement mechanism illustrated in part in FIGS. 1-3 and further in FIGS. 12a-12c. In FIGS. 1-3 and 12a-12c, the displacement mechanism is adapted to displace the outer structure 40 in an axial direction distally and proximally relative to the inner structure 20. In alternative embodiments (such as that illustrated in FIG. 15 as described further herein), the displacement mechanism is adapted to displace an inner structure in an axial direction proximally and distally relative to the outer structure. In FIGS. 1-3 and 12a-12c, the displacement mechanism includes a trigger line 100, as illustrated in FIGS. 12a-12c, adapted to pull the outer structure in an axial direction. Depending on the embodiment, a trigger line 100 may be used to pull the outer structure in a proximal direction, a distal direction, or both. The displacement mechanism may also include a spring 60. The spring 60 may be used to move the outer structure 40 in a distal or proximal direction. In the embodiments illustrated in FIGS. 1-3, a spring 60 biases the outer structure 40 in the distal direction.

Referring now to FIG. 3, a third embodiment is described. As illustrated in FIG. 3, each flexible engagement element 50 of the ligating band deployment structure or outer structure 40 may include a hinged tooth 80 for engaging the ligating bands 30 and at least one hinge 90 for attaching each hinged tooth 80 to the remainder of the outer structure 40. In an engagement position, the hinged tooth 80 extends radially inwardly to engage a ligating band 30 and push it forward. In this embodiment, each hinge 90 is configured to permit rotation of the hinged tooth 80 from its engagement position, such that when the outer structure 40 is moved proximally (or, in an alternative embodiment, when the inner structure is moved distally), the hinge 90 allows rotation of the hinged tooth 80 so that it can ride over a ligating band 30. Each hinge 90 is also configured to prevent rotation of the hinged tooth 80 from the engagement position when the outer structure 40 is moved distally so that when the outer structure 40 is moved distally the hinged tooth 80 forces the ligating band 30 distally over the outer surface of the inner structure 20.

The hinged tooth 80 and hinge 90 may be constructed in a variety of ways that will be apparent to persons skilled in the art. For example, the hinged tooth 80 may be configured to be operated by a spring mechanism that holds the hinged tooth 80 in a resting position disturbed only by the proximal movement of the outer structure 40. In alternative embodiments, the hinged tooth 80 may be configured to be controlled independently of the outer structure 40 such as, for example, by a separate trigger line or by automated electric means. In other alternative embodiments, the flexible engagement element of the ligating band deployment structure may comprise another flexible component, a component suspended from the remainder of the ligating band deployment structure, or a component attached to the remainder of the ligating band deployment structure by a “living hinge.”

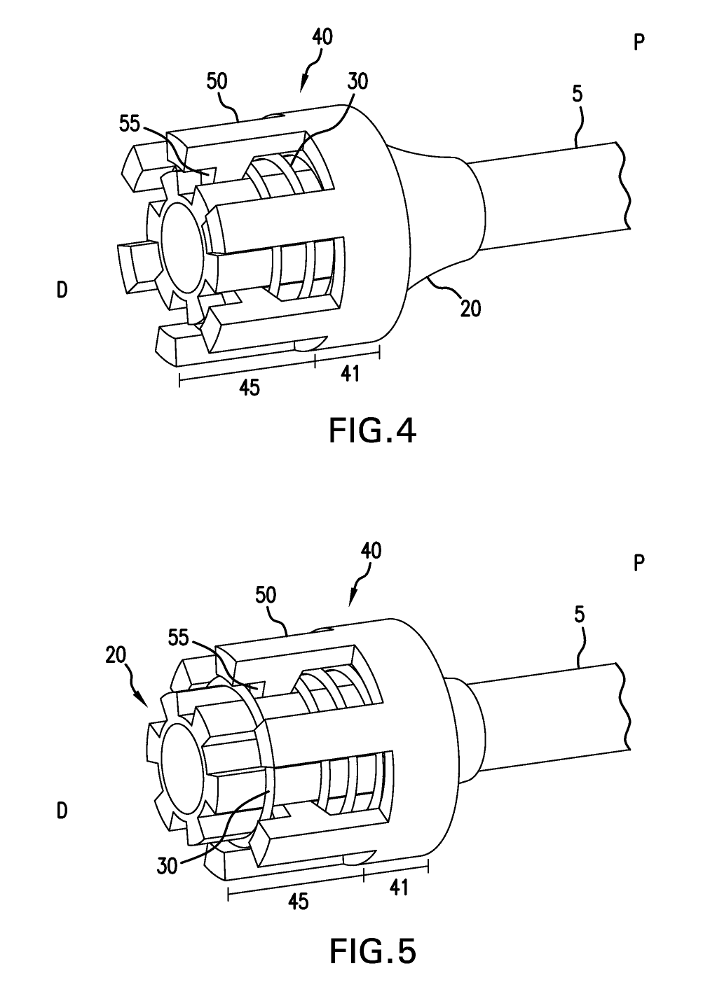

The operation of the embodiment of FIG. 1 is illustrated in FIGS. 4-7, but it will be understood that other embodiments, such as the embodiments of FIGS. 2 and 3, may operate in a similar manner. For the purpose of example only, the use of the ligating band dispenser 10 will be described with reference to the human esophagus. However, one of ordinary skill in the art will recognize that the ligating band dispenser 10 may be used throughout the human body where ligation is desired and may be accomplished using a device as disclosed.

After attaching the ligating band dispenser 10 to the distal end of an endoscope 5 via the flexible connector 6, the user, for example a physician, inserts the endoscope into the esophagus of the patient. The physician guides the endoscope 5 coupled with the ligating band dispenser 10 to find the distressed tissue, such as, for example, a varix on the esophageal surface. In this regard, it will be understood that the placement of the ligating band dispenser 10 on the endoscope 5 is such that the peripheral view of the endoscope is maximized as described in, for example, the Fortier '865 patent. It will be further understood that the inner structure 20 may be made of a transparent or translucent material so as to enhance the physician's view of the ligating bands 30 situated on the inner structure 20 and to enhance the overall peripheral vision of the endoscope.

It is contemplated that the outer cylinder 40 described in embodiments disclosed herein may also temporarily be moved to enhance the peripheral vision of the endoscope in certain situations. For example, the outer cylinder 40 may be retracted by the displacement mechanism so that the physician can obtain a better view.

Once the physician identifies a varix to be ligated, the physician moves the distal end of the inner structure 20 situated at the distal end of the endoscope 5 over the varix on the esophageal tissue. The varix may already be raised, or it may be enhanced with a fluid injection beneath the tissue. Once the distal end of the inner structure 20 is positioned over the varix, if desired, suction may be applied by any suitable means to force the distressed tissue into the channel 15 of the inner structure 20. The distal-most surface of the inner structure 20 may be a flat and substantially smooth surface, as illustrated in FIGS. 1-7, so as to provide a good surface for maintaining suction between the inner structure 20 and the esophageal tissue.

While disclosed embodiments describe a flat and smooth distal surface of inner structure 20, the surface of inner structure 20 may also be configured to further enhance suction, such as, for example, by treatment of the surface with a biochemical tissue adhesive or micro perforations for use in a more advanced vacuum system in addition to, or replacing, conventional endoscopic suction elements.

Up until this point, the ligating band dispenser 10 remains in a resting or start position, which may be as illustrated in FIG. 4. In the resting position illustrated in FIG. 4, the outer structure 40 is positioned such that the engagement surfaces 55 of the engagement elements 50 are distal to the first (i.e., the distal-most) ligating band 30. In alternative embodiments, the resting position can have the outer structure 40 positioned in other positions.

After suction has been established, the displacement mechanism is actuated to displace the outer structure 40 proximally into the first engagement position, as illustrated in FIG. 5. In the first engagement position, the engagement surfaces 55 of the engagement elements 50 are proximal to the first (i.e., the distal-most) ligating band 30, such that the first band 30 is engaged by the engagement surfaces 55. The resting position of the device may also be the first engagement position, such that the ligating band dispenser is initially provided in the position shown in FIG. 5.

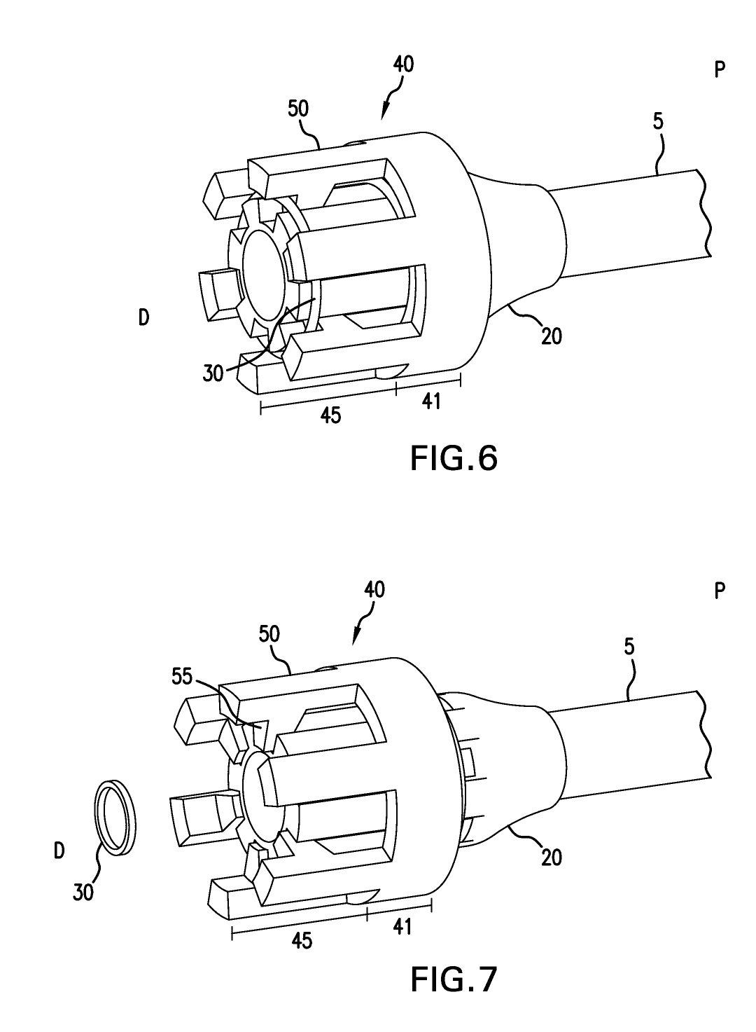

When the outer structure 40 is in the first engagement position, the first band 30 can be deployed by movement of the outer structure 40 distally to the deployment position. FIG. 6 shows the outer structure 40 moving from the first engagement position to the deployment position. When the outer structure 40 advances far enough distally, it pushes the ligating band off of the distal end of the inner structure 20 so that it deploys around the tissue. FIG. 7 shows the outer structure 40 in the deployment position, showing the ligating band deployed from the device.

After deployment of the first ligating band 30, the outer structure 40 may be refracted proximally to engage the next (second) ligating band 30. Accordingly, the displacement mechanism displaces the outer structure 40 proximally from the deployment position to the second engagement position to engage the second ligating band 30. Then, the displacement mechanism displaces the outer structure 40 distally from the second engagement position to the deployment position to deploy the second ligating band 30. Subsequent ligating bands 30 are deployed in a sequential manner, as described herein with respect to the first and second ligating bands, until all bands have been deployed or the procedure is otherwise terminated.

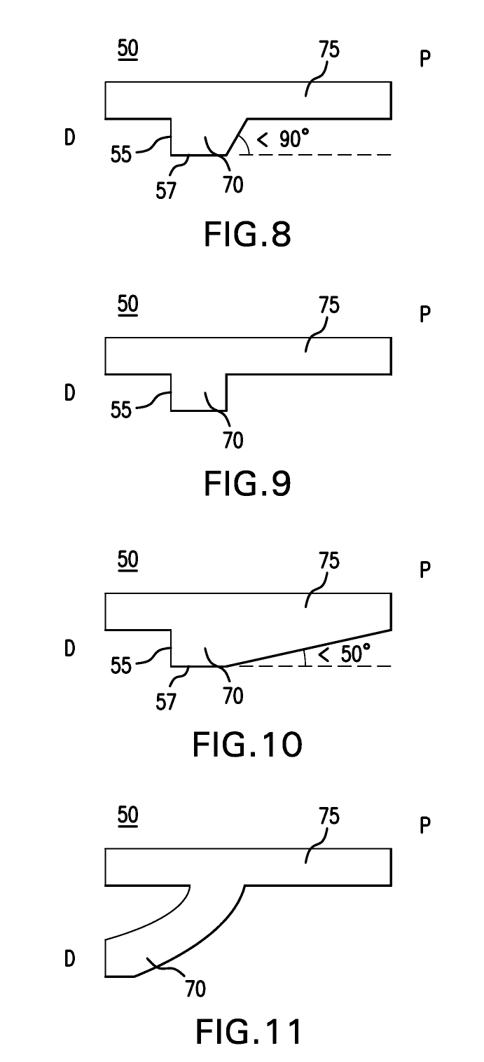

Referring again to the first and second embodiments as illustrated in FIGS. 1 and 2, the engagement structure 40 as shown in those figures includes at least one flexible engagement element 50, and each flexible engagement element 50 includes an engagement surface 55. FIGS. 8-11 illustrate alternative structures for the flexible engagement element 50. As illustrated in FIGS. 8-11, the flexible engagement element 50 includes a flexible arm 75 and finger 70 that protrudes inwardly in a radial direction toward the outer surface of the inner structure 20 from the flexible arm 75 to contact the inner structure 20. The distal-most surface of the finger 70 may be the engagement surface 55. The finger 70 may have an engagement surface 55 that extends substantially orthogonal to the longitudinal axis of the inner structure 20, as illustrated in FIG. 8.

The proximal surface of the finger 70, in certain embodiments, may be sloped from the bottom of the proximal surface to the top of the proximal surface. In the embodiment illustrated in FIG. 8, the angle of this slope relative to the inward radial-most surface 57 of the finger 70 is less than 90°, or it may have other configurations, such as less than 50°, as illustrated in FIG. 10. The sloping of this proximal surface facilitates movement of the flexible engagement element 50 over a ligating band when the outer structure 40 is displaced proximally. The sloping may be curved, straight, or any other suitable configuration. In other embodiments, the proximal-most surface of the finger 70 may be substantially orthogonal to the axial direction of the inner structure 20, similar to the distal-most surface illustrated in FIG. 9.

In other embodiments, each engagement surface 55 may be configured to the shape of the ligating band 30 to be deployed to enhance engagement of the flexible engagement element 50 with the ligating band 30 during deployment. FIG. 11 illustrates one such embodiment. In FIG. 11, the finger 70 is sloped distally in a radially inward direction from the flexible arm 75 so as to create a distal space between the finger 70 and the flexible arm 75 that fits the ligating band 30. Conforming the engagement element 50 in this manner enhances the contact between the engagement element 50 and the ligating band 30 to reduce disengagement during deployment. While the engagement surface of finger 70 in FIG. 11 that engages the ligating band 30 is illustrated as concave, an alternative design may be used in which the engagement surface is convex, which would allow the ligating band 30 to more easily slide off of the finger 70 for deployment. In FIG. 11, the proximal surface of the finger 70 is convex, which facilitates the movement of the finger 70 over the ligating bands when the ligating band deployment structure is moved in a proximal direction from the deployment position to an engagement position.

It is further contemplated that the inner structure 20 and/or the outer structure 40 may be coated with a lubricious coating, for example TEFLON (polytetrafluoroethylene), or treated with a lubricant to facilitate movement of the outer structure 40 across the outer surface of the inner structure 20 or to facilitate movement of the ligating bands across the surface of the inner structure 20.

While FIGS. 8-11 illustrate various forms of flexible engagement elements for the engagement structure of the outer structure, it will be appreciated that other forms are possible. For example, the engagement structure may comprise one or more flexible fingers or hinges that extend inwardly from a cylinder of the outer structure. Alternatively, the engagement structure may comprise one or more rigid arms, each having one or more flexible arms or hinges. Alternatively, the engagement structure may comprise one or more flexible arms, with or without fingers or hinges. Other configurations are possible within the scope of the invention.

FIGS. 12a-12c illustrate various forms for the displacement mechanism. In the embodiments illustrated in FIGS. 12a and 12c, the trigger line 100 is pulled proximally to displace the outer structure 40 proximally, and the spring 60 displaces the outer structure 40 distally. Thus, for example, the trigger line 100 is pulled to move the outer structure 40 from the resting position to the first engagement position. Then, the spring 60 displaces the outer structure 40 from the first engagement position to the deployment position to deploy the first band 30. Then, the trigger line 100 is pulled to displace the outer structure 40 from the deployment position to the second engagement position. Then, the spring 60 displaces the outer structure 40 from the second engagement position to the deployment position to deploy the second band 30. This action can be repeated to sequentially deploy the bands 30.

In alternative embodiments, such as illustrated in FIG. 12b, the trigger line 100 is pulled proximally to displace the outer structure 40 distally, and the spring 60 provides a proximal force on the outer structure 40 to displace the outer structure 40 proximally. Thus, for example, the trigger line 100 is pulled to displace the outer structure 40 from the first engagement position to the deployment position to deploy the first band 30. Then, the spring 60 displaces the outer structure proximally from the deployment position to the second engagement position. Next, the trigger line 100 is pulled to displace the outer structure 40 from the second engagement position to the deployment position to deploy the second band 30. This action can be repeated to sequentially deploy the bands 30.

The embodiments illustrated in FIGS. 12a-12c will now be described in more detail. As illustrated in FIG. 12a, the trigger line 100 extends inside outer channel 110, which runs along the outside of endoscope 5 axially from the proximal end to the distal end. The distal end of the trigger line 100 is mounted on the proximal end of outer structure 40 at the location marked by an “x.” The distal end of the trigger line 100 is mounted on the outer structure 40 by any suitable means. In this embodiment, pulling on the trigger line 100 creates a direct proximal force on the outer structure 40.

In the embodiment illustrated in FIG. 12b, the trigger line 100 extends through an inner channel 115, which runs through the inside of endoscope 5 axially from the proximal end to the distal end. The trigger line 100 then runs through the channel of the inner structure 20 and around the distal end of the inner structure 20. The trigger line 100 then returns in a proximal direction to be mounted to the proximal end of outer structure 40 at the location marked by an “x,” which is accomplished by any suitable means. The trigger line 100 runs under the ligating bands 30. In this example, the trigger line 100 runs through one of the plurality of tracks 25 to avoid entanglement with the ligating bands 30 during deployment. Trigger line 100 may further run through a slot (not shown) in the engagement surface 55 of the flexible engagement element 50 and underneath the engagement structure 45 to be mounted on the mounting portion 41 of the outer structure 40. Alternatively, a separate track not associated with an engagement element can be provided for the trigger line 100. In an alternative arrangement, the trigger line 100 in FIG. 12b, instead of being attached as shown, can extend further proximally, turn back distally around a hook or through a hole mounted at the proximal end of the inner structure 20 and then attach to the outer structure 40, such that pulling the trigger line 100 proximally pulls the outer structure proximally as in the embodiments illustrated in FIGS. 12a and 12c. With such an arrangement, the spring 60 can be arranged to force the outer structure 40 distally. Alternative configurations and placement of the trigger line 100 are possible within the scope of the invention, as would be understood by persons of ordinary skill in the art.

In the embodiment illustrated in FIG. 12c, the trigger line 100 extends through an inner channel 115, which runs through the inside of endoscope 5 axially from the proximal end to the distal end. The trigger line 100 then extends through an opening 125 in the inner structure 20 located between the distal end of the inner structure 20 and the distal end of the endoscope 5. The trigger line 100 is then mounted on the proximal end of outer structure 40 on the mounting portion 41 by any suitable means. The opening 125 may have a suitable seal so as not to adversely impact the suction through the inner structure 20.

In each of the embodiments illustrated in FIGS. 12a-12c, the force applied by the trigger line 100 and the spring 60 operate to balance and control movement of the outer structure 40. In this regard, such movement may be controlled by the operator from a proximal end of the endoscope outside the patient's body. When the trigger line 100 is used to move the outer structure 40 proximally and a spring 60 is used to push the outer structure 40 distally, the trigger line 100 may be pulled in increasing increments from the proximal end of the endoscope 5. That is, the trigger line 100 may be pulled a first distance to engage the first band 30, then released to allow the spring action to deploy the first band 30, then the trigger line 100 may be pulled a second distance greater than the first distance to engage the second band 30, then released to allow the spring action to deploy the second band 30, and so on. A gauge or controller may be provided at the proximal end to insure that the trigger line 100 is pulled the correct distance each time. When the trigger line 100 is used to move the outer structure 40 distally and a spring 60 is used to pull the outer structure 40 proximally, the trigger line 100 may be released in increasing increments from the proximal end of the endoscope 5. That is, the trigger line 100 may be released a first distance to allow the spring 60 to pull the outer structure 40 back proximally a first distance to engage the first band 30, then the trigger line 100 may be pulled to move the outer structure 40 distally to deploy the first band 30, then the trigger line 100 may be released a second distance greater than the first distance to allow the spring 60 to pull the outer structure 40 back proximally a second distance to engage the second band 30, then the trigger line 100 may be pulled to move the outer structure 40 distally to deploy the second band 30, and so on. A gauge or controller may be provided at the proximal end to insure that the trigger line 100 is released the correct distance each time.

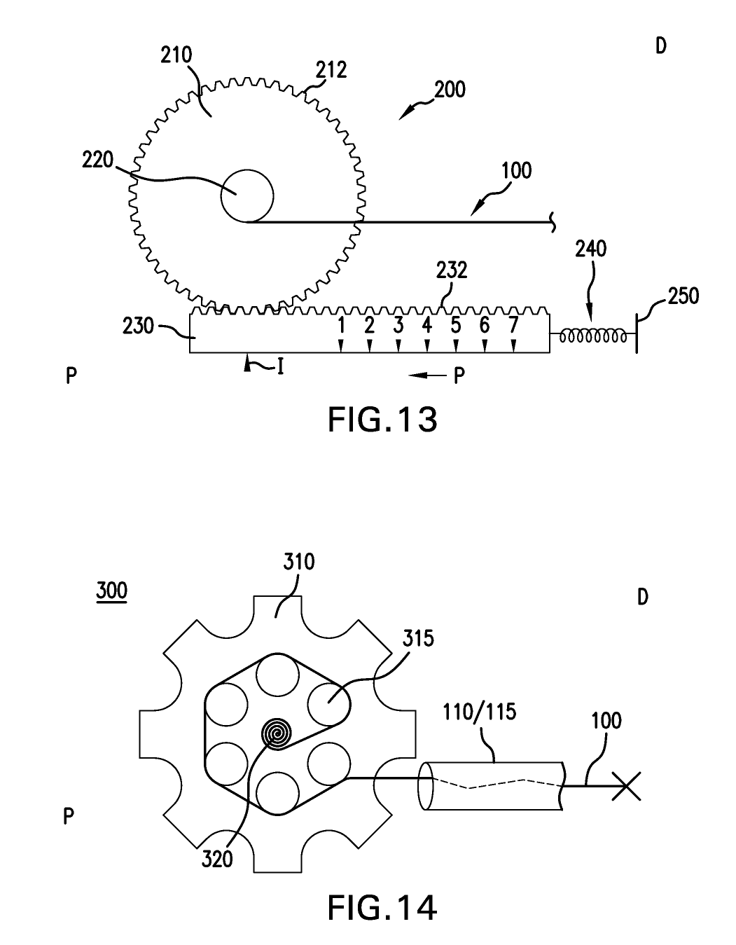

In alternative embodiments, the displacement mechanism may include an actuator 200 or 300 located at the proximal end of the endoscope 5 for pulling the trigger line 100 in a proximal direction in sequential increments so as to cause the engagement structure 50 of the outer structure 40 to sequentially engage each of the ligating bands 30 for sequential deployment of each of the ligating bands 30 from the distal end of the inner structure 20, as illustrated in FIGS. 13 and 14.

In the embodiment illustrated in FIG. 13, the actuator 200 comprises a rack and pinion system for controlling movement of the outer structure 40 in the axial direction. For purposes of this embodiment, the actuator 200 is described relative to the embodiment of the displacement mechanisms illustrated in FIGS. 12a and 12c. It will be understood, however, that this embodiment could be configured to be used with other displacement mechanisms.

As illustrated in FIG. 13, an exemplary rack and pinion system includes a pinion 210 and a rack 230. The rack 230 has teeth 232 that engage teeth 212 on pinion 210. The pinion 210 is coupled to a spool 220 on which the trigger line 100 can be wound. The rack is coupled to a spring 240 which in turn is attached to a housing 250 (shown schematically). The pinion 210 can be mounted with respect to the housing 250 for rotation within the housing 250. The rack 230 can be mounted with respect to the housing 250 for sliding back and forth within the housing 250.

In the embodiment of FIG. 13, the housing 250 can have an index mark I, and the rack can have a set of indicia as shown, such as the numbers 1 through 7, corresponding to the engagement positions for the ligating bands 30. To move the outer structure 40 to the first engagement position, the physician moves the rack 230 in the proximal direction (shown by the arrow P) until the indicia “1” lines up with the index mark I. This rotates the pinion 210 by the proper distance so as to pull the trigger wire 100 proximally the correct amount in order to move the outer structure proximally to the first engagement position. Then, upon release of the rack 230, the spring action of spring 240 returns the rack 230, and the spring action of the spring 60 moves the outer structure 40 to the deployment position, deploying the first band. Then, to move the outer structure 40 to the second engagement position, the physician moves the rack 230 in the proximal direction until the indicia “2” lines up with the index mark I. Then, upon release, the spring action of spring 240 returns the rack 230, and the spring action of the spring 60 moves the outer structure 40 to the deployment position, deploying the second band. This action is repeated to sequentially deploy the bands.

The rack 230 may be attached to a suitable finger trigger or other mechanism to allow it to be actuated. In addition, the rack may cooperate with a stop mechanism that sequentially stops the rack 230 at each sequential stopping place for the sequential bands. For example, the rack 230 can have a pin that cooperates with a series of slots, the length of each slot corresponding to the distance required to move to the engagement position for a respective band 30. Upon first retraction of the rack 230, the pin moves within the first slot to move the rack 230 to the first engagement position. Then, upon release, the pin moves to the second slot, such that upon second retraction of the rack 230, the pin moves within the second slot, longer than the first, so as to move the rack 230 to the second engagement position. Various other mechanisms may be used to insure retraction of the rack 230 by the correct distance each time. For example, the retraction distance may be computer controlled.

In another embodiment, illustrated in FIG. 14, the actuator 300 may comprise a disk and peg system for controlling movement of the outer structure 40 in the axial direction. For purposes of this embodiment, the actuator 300 is described relative to the embodiment of the displacement mechanism illustrated in FIGS. 12a and 12c. It will be understood, however, that this embodiment could be configured to be used with other displacement mechanisms.

As illustrated in FIG. 14, an exemplary disk and peg system includes a corrugated disk 310 that includes breakable pegs 315 and spool 320. FIG. 14 also depicts trigger line 100 running through outer channel 110 or inner channel 115 of the endoscope 5, a distal end of the trigger line 100 being mounted on the proximal end of outer structure 40. In this embodiment, the trigger line 100 is configured to be fixed on the spool 320 and wrapped around the radial-most surfaces of the each breakable peg 315. Although fixed to the corrugated disk 310, breakable pegs 315 are configured to hold the trigger line 100 taut against the spring 60 acting on the outer structure 40 until the point of deployment, whereupon the increasing distal pushing force of the spring 60 acting on the outer structure 40 increases to a point at which it breaks the breakable peg 315. The breakable pegs 315 can be made of any suitable material with a predetermined strength high enough to resist the distal force of the spring to the point before deployment but low enough to break under the increased distal force from the spring 60 as the corrugated disk 310 is actuated.

In practice, for example, when the engagement structure 45 is in a resting position, the physician actuates the corrugated disk 310 in a clockwise direction pulling the trigger line 100 in a proximal direction, which moves the outer structure proximally, against the distal counteractive force of the spring 60, into the first engagement position. As the corrugated disk 310 is actuated, the counteractive distal force from the spring 60 on the trigger line 100 increases such that once the outer structure 40 reaches the engagement position, as shown in FIG. 5, the distal force from the spring 60 acting on the outer structure 40 and thus the trigger line 100 provides a high enough force to break the clockwise-most breakable peg 315. Under this force, the clockwise-most breakable peg 315 breaks, disengaging the trigger line 100 from the clockwise-most breakable peg 315 and creating slack in the trigger line 100. With slack in the trigger line 100, the distal force of the spring 60 moves the outer structure 40 distally into the deployment position, as shown in FIG. 6. To move the outer structure into the second engagement position, the physician again actuates the corrugated disk 310 in a clockwise direction. The slack in the trigger line 100 remaining from the previous deployment is spooled around spool 320 until the trigger line is again taut against the next clockwise-most breakable peg 315. The next clockwise-most breakable peg 315 is slightly stronger that the first one, such that the outer structure 40 can be pulled farther back, to the second engagement position engaging the second ligating band, before the second peg breaks. Once the outer structure 40 reaches the second engagement position, the force from the spring 60 breaks the second peg, and the spring forces the outer structure 40 to the deployment position, deploying the second band. It will be appreciated that each successive peg is slightly stronger than the one before it, to allow the outer structure 40 to be pulled back to sequentially increasing distances, in order to reach each of the ligating bands 30 in succession.

While the illustrated embodiments show a manually operated trigger line 100, it is also envisioned that the trigger line 100 may be replaced by or part of an automated system for applying the appropriate force to pull the outer structure 40 distally or proximally. For example, the force to move the outer structure 40 may be supplied by electrical actuation, hydraulic actuation, heat actuation, shape memory material actuation, or electroactive material actuation (e.g., metals, polymers, gel). Similarly, the spring 60 may also be replaced by or part of an automated system for applying the compressive or distal force, such as, for example, an electric or hydraulic pump, or other forms of electrical actuation, hydraulic actuation, heat actuation, shape memory material actuation, or electroactive material actuation. In one example embodiment in which the displacement mechanism does not include a trigger line, the ligating band support structure and the ligating band deployment structure may each be attached to a tube, one inside the other, with the tubes moveable with respect to each other to move the device between the engagement positions and the deployment position. Other constructions for the displacement mechanism and its actuation are possible. For example, the displacement mechanism may include a handle and trigger arrangement, a dial mechanism and ratchet, or other suitable displacement mechanisms that are currently known in the art.

As illustrated in FIG. 15, a ligating band dispenser 10 according to another embodiment may include features of the embodiments disclosed herein except that the outer structure 40 is mounted on the endoscope and the inner structure 20 is moved proximally and distally by the displacement mechanism. In this embodiment, the displacement mechanism displaces the inner structure 20 proximally from the first engagement position to the deployment position to deploy the first ligating band 30, the displacement mechanism displaces the inner structure 20 distally from the deployment position to the second engagement position to engage the second ligating band 30, and the displacement mechanism displaces the inner structure 20 proximally from the second engagement position to the deployment position to deploy the second ligating band 30, and so on. Compared to the embodiments depicted in FIGS. 1-3 with a spring 60 and mounting portion 41, as illustrated in FIG. 15, the mounting portion 41 is extended proximally to mount the outer structure 40 to the endoscope.

In this embodiment, an alternative seal structure (not shown) may be provided for maintaining appropriate suction while the inner structure 20 is moved relative to the outer structure 40. While FIG. 15 illustrates the inner structure 20 as a sliding structure, alternative configurations for the inner structure 20 are possible, such as, for example, a collapsing or telescoping structure.

While embodiments such as those illustrated in FIGS. 1-3 and 15 have the ligating band deployment structure as an outer structure that is positioned outside the outer surface of the ligating band support structure, in alternative embodiments the ligating band deployment structure may be positioned radially inside of, or underneath, the ligating bands. Thus, for example, the ligating band support structure may have one or more tracks or grooves beneath the ligating bands for accommodating one or more engagement elements of the ligating band deployment structure. The engagement elements may be in the form of arms, fingers, or teeth as disclosed herein. The arms, fingers, or teeth may project radially outwardly between the ligating bands to advance the ligating bands relative to the ligating band support structure similar to other embodiments described herein. The displacement and actuation of the components may be similar to other embodiments as described herein.

Disclosed embodiments have been described with reference to several exemplary embodiments. There are many modifications of the disclosed embodiments which will be apparent to those of skill in the art. It is understood that these modifications are within the teaching of the present invention which is to be limited only by the claims.

What is claimed is:

1. A ligating band dispenser device for mounting on a distal end of an endoscope comprising:
a ligating band support structure having a longitudinal axis, an outer surface, a proximal end and a distal end, the ligating band support structure being adapted to be mounted on the distal end of the endoscope;
at least one ligating band mounted around the outer surface of the ligating band support structure;
a ligating band deployment structure, the ligating band deployment structure having an engagement structure for engaging and deploying the at least one ligating band; and
a displacement mechanism adapted to displace the ligating band deployment structure in an axial direction distally and proximally relative to the ligating band support structure;
wherein the ligating band deployment structure has a first engagement position in which the engagement structure engages a first ligating band, and a deployment position distal to the first engagement position in which the engagement structure causes the engaged ligating band to deploy from the distal end of the ligating band support structure.

2. The ligating band dispenser according to claim 1, wherein the ligating band support structure is an inner structure, the ligating band deployment structure is an outer structure, and the outer structure is positioned outside the outer surface of the inner structure.

3. The ligating band dispenser according to claim 1, wherein the ligating band dispenser device comprises a plurality of ligating bands mounted around the outer surface of the ligating band support structure.

4. The ligating band dispenser according to claim 3, wherein the ligating band deployment structure further has a second engagement position in which the engagement structure engages a second ligating band; and
wherein the displacement mechanism displaces the ligating band deployment structure distally from the first engagement position to the deployment position to deploy the first ligating band, the displacement mechanism displaces the ligating band deployment structure proximally from the deployment position to the second engagement position to engage the second ligating band, and the displacement mechanism displaces the ligating band deployment structure distally from the second engagement position to the deployment position to deploy the second ligating band.

5. The ligating band dispenser according to claim 4, wherein the engagement structure engages and moves the first ligating band when the displacement mechanism displaces the ligating band deployment structure distally from the first engagement position to the deployment position to deploy the first ligating band, wherein the engagement structure moves relative to but does not move the second ligating band when the displacement mechanism displaces the ligating band deployment structure proximally from the deployment position to the second engagement position to engage the second ligating band, and wherein the engagement structure engages and moves the second ligating band when the displacement mechanism displaces the ligating band deployment structure distally from the second engagement position to the deployment position to deploy the second ligating band.

6. The ligating band dispenser device according to claim 5, wherein the engagement structure comprises at least one flexible engagement element, each flexible engagement element comprising an engagement surface for sequentially engaging each of the ligating bands and for sequentially pushing each of the ligating bands distally to sequentially deploy each of the ligating bands from the distal end of the ligating band support structure.

7. The ligating band dispenser device according to claim 6, wherein each engagement surface is configured to engage the ligating band to be deployed to enhance engagement of the flexible engagement element with the ligating band during deployment.

8. The ligating band dispenser device according to claim 6, wherein each engagement surface extends substantially orthogonal to the longitudinal axis of the ligating band support structure.

9. The ligating band dispenser device according to claim 6, wherein each flexible engagement element further comprises one of a sloped or curved proximal surface adapted to facilitate movement of the flexible engagement element over a ligating band when the ligating band deployment structure is displaced proximally.

10. The ligating band dispenser device according to claim 6, wherein each flexible engagement element comprises a flexible arm.

11. The ligating band dispenser device according to claim 6, wherein each flexible engagement element comprises a finger extending inwardly in a radial direction toward the outer surface of the ligating band support structure.

12. The ligating band dispenser device according to claim 6, wherein each flexible engagement element comprises a hinged tooth for engaging the ligating bands and at least one hinge for attaching each hinged tooth to the remainder of the ligating band deployment structure.

13. The ligating band dispenser device according to claim 6, wherein the outer surface of the ligating band support structure comprises at least one track extending in the axial direction and corresponding to the at least one flexible engagement element.

14. The ligating band dispenser device according to claim 1, wherein the displacement mechanism comprises:
a trigger line adapted to pull the ligating band deployment structure in a proximal direction; and
a spring biasing the ligating band deployment structure in a distal direction.

15. The ligating band dispenser device according to claim 14, wherein the trigger line extends axially through a channel inside the endoscope and is fixed to the ligating band deployment structure to pull the ligating band deployment structure in a distal direction.

16. The ligating band dispenser device according to claim 14, wherein the trigger line extends axially through a channel inside the endoscope and through an opening in the ligating band support structure located between the distal end of the ligating band support structure and the distal end of the endoscope and is fixed to the ligating band deployment structure to pull the ligating band deployment structure in a proximal direction.

17. The ligating band dispenser device according to claim 14, wherein the trigger line extends axially outside the endoscope and is fixed to the ligating band deployment structure to pull the ligating band deployment structure in a proximal direction.

18. The ligating band dispenser device according to claim 3, wherein the displacement mechanism comprises:
a trigger line adapted to pull the ligating band deployment structure in a proximal direction; and
an actuator located at the proximal end of the endoscope for pulling the trigger line in a proximal direction in sequential increments to cause the engagement structure of the ligating band deployment structure to sequentially engage each of the ligating bands for sequential deployment of each of the ligating bands from the distal end of the ligating band support structure.

19. The ligating band dispenser device according to claim 18, wherein the actuator comprises:
a pinion gear attached to the trigger line; and
a rack gear coupled to the pinion gear and adapted to be moved linearly in increasing sequential increments to rotate the pinion gear in increasing sequential increments to cause the engagement structure of the ligating band deployment structure to sequentially engage each of the ligating bands for sequential deployment of each of the ligating bands from the distal end of the ligating band support structure.

20. The ligating band dispenser device according to claim 18, wherein the actuator comprises:
a disk attached to the trigger line; and
a plurality of breakable pegs mounted on the disk;
wherein the disk is adapted to be rotated in increasing sequential increments by the breaking of one of the breakable pegs after each increment; and
wherein the rotation of the disk in increasing sequential increments causes the engagement structure of the ligating band deployment structure to sequentially engage each of the ligating bands for sequential deployment of each of the ligating bands from the distal end of the ligating band support structure.

21. A ligating band dispenser device for mounting on a distal end of an endoscope comprising:
a ligating band support structure having a longitudinal axis, an outer surface, a proximal end and a distal end;
at least one ligating band mounted around the outer surface of the ligating band support structure;
a ligating band deployment structure, the ligating band deployment structure being adapted to be mounted on the distal end of the endoscope, the ligating band deployment structure having an engagement structure for engaging and deploying the at least one ligating band; and
a displacement mechanism adapted to displace the ligating band support structure in an axial direction distally and proximally relative to the ligating band deployment structure;
wherein the ligating band support structure has a first engagement position in which the engagement structure engages a first ligating band, and a deployment position proximal to the first engagement position in which the engagement structure causes the engaged ligating band to deploy from the distal end of the ligating band support structure.

22. The ligating band dispenser according to claim 21, wherein the ligating band support structure is an inner structure, the ligating band deployment structure is an outer structure, and the outer structure is positioned outside the outer surface of the inner structure.

23. The ligating band dispenser according to claim 21, wherein the ligating band dispenser device comprises a plurality of ligating bands mounted around the outer surface of the ligating band support structure.

24. The ligating band dispenser according to claim 23, wherein the ligating band support structure further has a second engagement position in which the engagement structure engages a second ligating band; and
wherein the displacement mechanism displaces the ligating band support structure proximally from the first engagement position to the deployment position to deploy the first ligating band, the displacement mechanism displaces the ligating band support structure distally from the deployment position to the second engagement position to engage the second ligating band, and the displacement mechanism displaces the ligating band support structure proximally from the second engagement position to the deployment position to deploy the second ligating band.

25. The ligating band dispenser according to claim 24, wherein the engagement structure engages and moves the first ligating band relative to the ligating band support structure when the displacement mechanism displaces the ligating band support structure proximally from the first engagement position to the deployment position to deploy the first ligating band, wherein the engagement structure does not move the second ligating band when the displacement mechanism displaces the ligating band support structure distally from the deployment position to the second engagement position to engage the second ligating band, and wherein the engagement structure engages and moves the second ligating band relative to the ligating band support structure when the displacement mechanism displaces the ligating band support structure proximally from the second engagement position to the deployment position to deploy the second ligating band.

26. The ligating band dispenser device according to claim 25, wherein the engagement structure comprises at least one flexible engagement element, each flexible engagement element comprising an engagement surface for sequentially engaging each of the ligating bands and for sequentially pushing each of the ligating bands distally to sequentially deploy each of the ligating bands from the distal end of the ligating band support structure.

27. The ligating band dispenser device according to claim 23, wherein the displacement mechanism comprises:
a trigger line adapted to pull the ligating band support structure in a proximal direction; and
a spring biasing the ligating band support structure in a distal direction.

28. A method of deploying a plurality of ligating bands, comprising:
(a) providing a ligating band dispensing device comprising:
(i) a ligating band support structure having a longitudinal axis, an outer surface, a proximal end and a distal end;
(ii) a plurality of ligating bands mounted around the outer surface of the ligating band support structure;
(iii) a ligating band deployment structure, the ligating band deployment structure having an engagement structure for sequentially engaging and deploying each of the ligating bands; and
(iv) a displacement mechanism adapted to displace one of the ligating band deployment structure and ligating band support structure in an axial direction distally and proximally relative to the other of the ligating band deployment structure and ligating band support structure;
(b) positioning the ligating band dispensing device in a first engagement position in which the engagement structure of the ligating band deployment structure engages a first ligating band mounted around the outer surface of the ligating band support structure;
(c) actuating the ligating band dispensing device from the first engagement position to a deployment position to deploy the first ligating band from the distal end of the ligating band support structure;
(d) positioning the ligating band dispensing device in a second engagement position in which the engagement structure of the ligating band deployment structure engages a second ligating band mounted around the outer surface of the ligating band support structure; and
(e) actuating the ligating band dispensing device from the second engagement position to the deployment position to deploy the second ligating band from the distal end of the ligating band support structure.
